(19) United States
(12) Patent Application Publication
(10) Pub. No.: US 2024/0226435 A9
(43) Pub. Date: Jul. 11, 2024

(54) Low Dead Volume Adaptor for a Syringe

(71) Applicant: Becton, Dickinson and Company, Franklin Lakes, NJ (US)

(72) Inventors: Suzanne Ferreri, Glen Rock, NJ (US); Paul Paia Marici, Piscataway, NJ (US)

(21) Appl. No.: 17/969,903

(22) Filed: Oct. 20, 2022

(51) Int. Cl. A61M 5/178

(52) U.S. Cl. CPC A61M 5/178; A61M 5/3293

(57) ABSTRACT

An adaptor for a syringe including a shaft with a lumen and a seal. The shaft is sized and shaped to be inserted in the lumen of the syringe tip and create a seal with the opening of the syringe tip, and the seal is sized and shaped to be inserted into the needle hub and create a seal with the needle hub, such that the adaptor reduces the interior volume of the lumen of the syringe tip and the needle hub that is filled with medicament contained in the syringe. Also, an adaptor assembly comprising the adaptor and a delivery housing to which the adaptor is attached, a syringe body including the adaptor, a needle assembly including the adaptor, and a syringe including the adaptor. Further, a method of reducing the dead volume of a syringe in which the adaptor is provide in the syringe as described above.

### BACKGROUND OF THE INVENTION

### Field of the Invention

The present invention is directed to an adaptor that reduces the dead volume of a standard syringe, a syringe body including the adaptor, a needle hub including the adaptor, and a syringe including the adaptor, as well as a method of reducing the dead volume of a syringe utilizing the adaptor.

### Description of Related Art

Fittings and hubs for attaching needles to syringe bodies have been standardized, for example, under ISO-80369-7:2016, revised in 2021 entitled “Small-bore connectors for liquids and gases in healthcare applications—Part 7: Connectors for intravascular or hypodermic applications”. As can be seen in FIG. 1, in such standardized syringes that utilize needle assemblies having hubs that fit over a male luer taper, an area in which a significant amount of medicament is retained after an injection using the syringe is present in the lumen of the luer taper and in the needle hub. This volume of medicament, as well as the medicament that is retained in the lumen of the needle, is referred to as the dead volume. Thus, standardized syringes have a significant dead volume which results in a significant amount of medicament being wasted with each injection and a reduced number of doses being able to be obtained from a vial of medicament having a specific volume, thereby increasing packaging and storage costs. For many high value or rare drugs, this unintended medication waste can limit access to a drug and/or increase the cost.

Therefore, it would be advantageous to reduce the dead volume of standardized syringes in order to reduce the amount of medicament that is wasted and to reduce packaging and storage costs, especially when the medicament must be refrigerated.

### SUMMARY OF THE INVENTION

The present invention is directed to an adaptor for a syringe having a syringe tip defining a lumen with an opening and a needle assembly having a needle hub attached to the syringe tip. The adaptor comprises a shaft having a proximal end, a distal end, and a sidewall extending between the proximal end and the distal end and defining a lumen extending through the shaft and a seal that extends from the distal end of the shaft. At least a portion of the shaft is sized and shaped to be inserted in the lumen of the syringe tip and create a seal with the opening of the syringe tip, and at least a portion of the seal is sized and shaped to be inserted into the needle hub of the needle assembly to create a seal with an inner surface of the needle hub, thereby reducing an interior volume of the lumen of the syringe tip and an interior volume of the needle hub that is filled with medicament contained in the syringe.

The seal may surround the distal end of the shaft and has a proximal end, a distal end, and a sidewall. An outer surface of the sidewall of the seal may taper in the distal direction with a largest outer diameter of the seal at the proximal end of the seal and a smallest outer diameter of the seal at the distal end of the seal.

The seal may be a circumferential flange extending from the distal end of the shaft. The seal may have a concave curvature or the outer diameter of the seal may taper in a distal direction, such that a largest diameter of the seal is at a proximal end of the seal, a smallest diameter of the seal is at a distal end of the seal, and the seal extends in a proximal direction along the shaft. The seal may have a convex curvature or the outer diameter of the seal may taper in a proximal direction, such that the largest diameter of the seal is at a distal end of the seal, the smallest diameter of the seal is at a proximal end of the seal, and the seal extends away from the distal end of the shaft.

The shaft and/or the seal may be made of a material which compresses or flexes when the shaft is inserted into the opening of the syringe tip or the seal is inserted in the needle hub, respectively.

A sealing surface of the seal may have the shape of a cone, a truncated cone, a hemisphere, or a cylinder with the front portion having a flat seal surface.

A diameter of the lumen of the shaft may change along the length of the adaptor. A distal portion of the lumen of the shaft that is surrounded by the seal and positioned in the needle hub may have a diameter that is greater than a diameter of a proximal portion of the shaft that is inserted in the lumen of the syringe tip.

A distal portion of the shaft that is surrounded by the seal and positioned in the needle hub has an outer diameter that is greater than an outer diameter of a proximal portion of the shaft that is inserted in the lumen of the syringe tip.

The present invention is also directed to an adaptor assembly comprising an adaptor as described above and a delivery housing to which the adaptor is attached.

The delivery housing may comprise an open proximal end, a closed distal end, and a sidewall extending between the proximal end and the distal end.

An inner surface of the sidewall of the delivery housing at the closed distal end of the delivery housing has a diameter equal to or less than a maximum diameter of the seal, such that the adaptor is held within the delivery housing by frictional and/or compressive forces between the inner surface of the sidewall of the delivery housing and the seal. An inner sidewall of the delivery housing may have a recess having a shape corresponding to a shape of the outer surface of the seal. Frictional and/or compressive forces between the adaptor and the delivery housing may be less than the frictional and/or compressive forces between the adaptor and the syringe tip.

A post may extend proximally outward from an inner surface of the closed proximal end of the delivery housing with the post being inserted in the lumen of the shaft of the adaptor or a protrusion comprising a sidewall defining a passageway may extend proximally outward from an inner surface of the closed proximal end of the delivery housing with the shaft of the adaptor being inserted in the passageway of the protrusion. Frictional and/or compressive forces between the adaptor and the delivery housing may be less than the frictional and/or compressive forces between the shaft of the adaptor and the post and the shaft of the adaptor and the protrusion.

A flange adapted to engage threads of a luer lock on the syringe may extend radially outward from the open distal end of the delivery housing or the delivery housing may include threads on the inner surface of the sidewall.

The delivery housing may include ridges extending radially outward from an outer surface of the sidewall.

The present invention is also directed to a syringe body comprising a chamber having a syringe tip defining a lumen having an opening through which medicament may be expelled from the chamber and an adaptor as described above. At least a portion of the shaft of the adaptor is positioned in the lumen of the syringe tip and forms a seal with the opening of the lumen. A length of the shaft that is inserted into the lumen of the syringe tip may be substantially equal to a length of the lumen of the syringe tip.

The present invention is also directed to a needle assembly comprising a needle hub having a sidewall defining an interior volume, a needle having a lumen in fluid communication with the interior volume of the needle hub and extending from the needle hub, and an adaptor as described above. The seal of the adaptor is positioned in the interior volume of the needle hub and forms a seal with an inner surface of the sidewall of the needle hub.

The present invention is also directed to a syringe comprising a syringe body comprising a chamber having a syringe tip defining a lumen having an opening through which medicament may be expelled from the chamber, a needle hub having a sidewall defining an interior volume connected to the syringe tip, a needle having a lumen in fluid communication with the interior volume of the needle hub and extending from the needle hub, and an adaptor as described above. At least a portion of the shaft of the adaptor is positioned in the lumen of the syringe tip and forms a seal with the opening of the lumen, the seal of the adaptor is positioned in the interior volume of the needle hub and forms a seal with an inner surface of the sidewall of the needle hub, and the lumen of the shaft of the adaptor is in fluid communication with the chamber and the lumen of the needle. At least a portion of the shaft of the adaptor may fill part of a volume of the lumen of the syringe tip and the seal of the adaptor may fill and isolate a portion of the interior volume of the needle hub that would otherwise be in filled with the medicament. A length of the shaft that is inserted into the lumen of the syringe tip may be substantially equal to a length of the lumen of the syringe tip.

The present invention is also directed to a method of reducing the dead volume of a syringe having a syringe tip defining a lumen with an opening and a needle assembly having a needle hub attached to the syringe tip. The method comprises providing an adaptor as described above, inserting at least a portion of the shaft of the adaptor into the lumen of the syringe tip and inserting the seal of the adaptor in an interior volume of the needle hub. A seal is provided between the shaft of the adaptor and the opening of the syringe tip, and a seal is provided between the seal of the adaptor and an inner surface of the needle hub, and the adaptor reduces an interior volume of the lumen of the syringe tip and the interior volume of the needle hub that is filled with medicament contained in the syringe.

### BRIEF DESCRIPTION OF THE DRAWINGS

FIG. 1 is a cross-sectional view of a proximal end of a prior art syringe;

FIG. 2 is a side view of a first embodiment of an adaptor according to the invention;

FIG. 3 is a cross-sectional view of the adaptor of FIG. 2;

FIG. 4 is a side view of a second embodiment of an adaptor according to the invention;

FIG. 5 is a cross-sectional view of the adaptor of FIG. 4;

FIG. 6 is a side view of a first embodiment of an adaptor according to the invention;

FIG. 7 is a cross-sectional view of the adaptor of FIG. 6;

FIG. 8 is perspective view of an adaptor housing according to the invention;

FIG. 9 is a cross-sectional view of a first embodiment of an adaptor assembly according to the invention with the adaptor of FIGS. 2 and 3 according to the invention;

FIG. 10 is a cross-sectional view of the adaptor assembly of FIG. 9 with the adaptor of FIGS. 6 and 7 according to the invention;

FIG. 11 is a cross-sectional view of a second embodiment of an adaptor assembly with the adaptor of FIGS. 4 and 5 according to the invention;

FIG. 12 is a cross-sectional view of a third embodiment of an adaptor assembly with the adaptor of FIGS. 2 and 3 according to the invention;

FIG. 13 is a cross-sectional view of a fourth embodiment of an adaptor assembly with the adaptor of FIGS. 2 and 3 according to the invention;

FIGS. 14A-14C are cross-sectional views of an adaptor according to the invention being inserted into a syringe body using an adaptor assembly according to the invention;

FIG. 15 is a cross-sectional view of a proximal end of a syringe body of the invention with the inventive adaptor of FIGS. 2 and 3;

FIG. 15 is a cross-sectional view of a needle assembly of the invention with the adaptor of FIGS. 2 and 3;

FIG. 16 is a cross-sectional view of a proximal end of a syringe of the invention with the inventive adaptor of FIGS. 2 and 3; and

FIG. 17 is a cross-sectional view of the syringe body and needle hub.

### DESCRIPTION OF THE INVENTION

As used herein, any numerical values are expressed using a period as a decimal point and a comma as a thousand separator, for example, 1,234 would be one thousand two hundred thirty four, and 1.2 would be one and two tenths. Unless otherwise expressly specified, all numbers, such as those expressing values, ranges, amounts or percentages, may be read as if prefaced by the word “about”, even if the term does not expressly appear. Any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1. Plural encompasses singular and vice versa. When ranges are given, any endpoints of those ranges and/or numbers within those ranges can be combined within the scope of the present invention. “Including”, “such as”, “for example”, and like terms mean “including/such as/for example but not limited to”.

For purposes of the description hereinafter, spatial orientation terms, for example, “upper”, “lower”, “right”, “left”, “vertical”, “horizontal”, “top”, “bottom”, “lateral”, “longitudinal”, “proximal”, “distal” and derivatives thereof, relate to the invention as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

As referred to herein, “proximal” refers to the end of the catheter adaptor 10 and the direction indicated by the arrow “P” in the figures and “distal” refers to the end of the catheter adaptor 10 and the direction indicated by the arrow “D” in the figures.

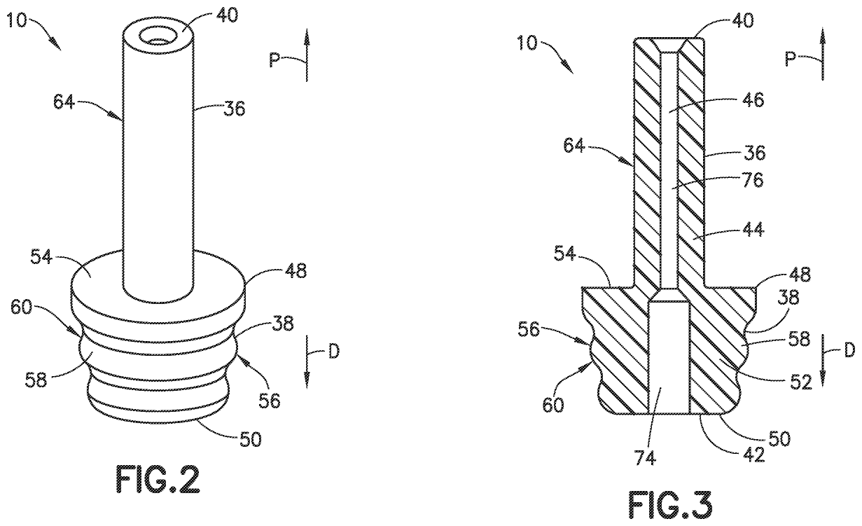

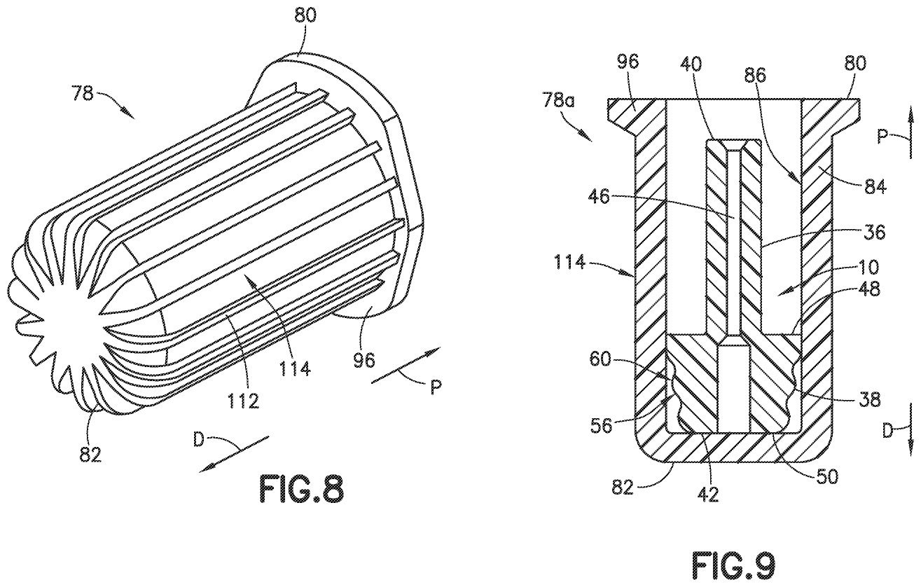

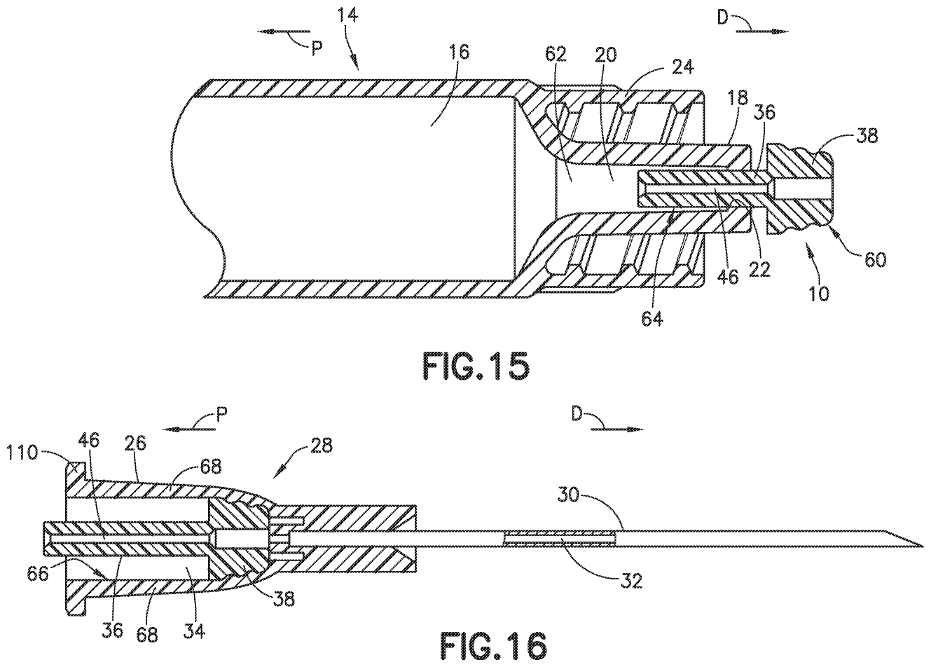

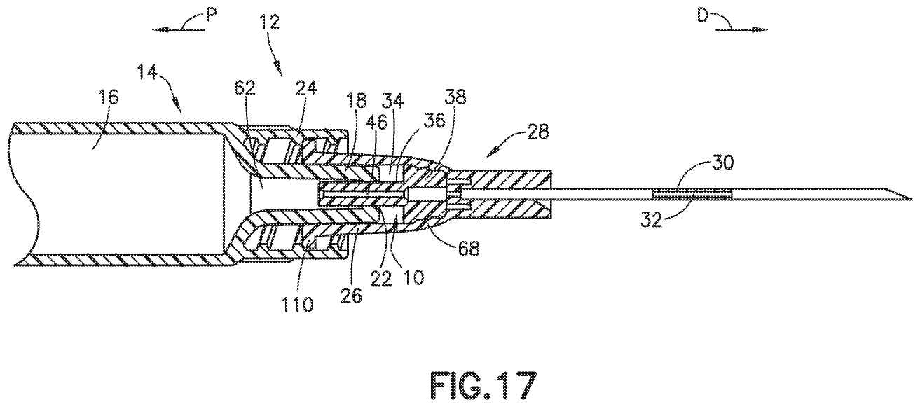

The present invention is directed to a low dead volume adaptor for a syringe (FIGS. 2-7), which can include a delivery housing (FIGS. 8-14C), a syringe body comprising a low dead volume adaptor (FIG. 15), a needle hub comprising a low dead volume adaptor (FIG. 16), and a syringe comprising a low dead volume adaptor (FIG. 17).

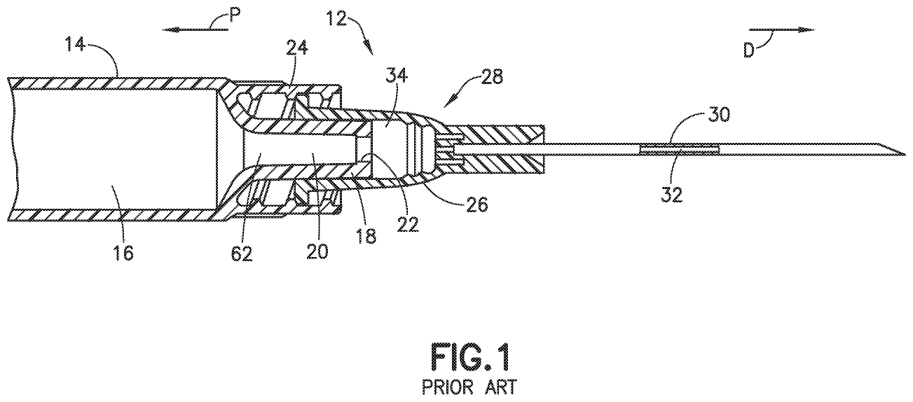

The adaptor 10, 10a, 10b is used with standard syringes and needle assemblies, for example, ISO compliant syringes or needle assemblies having connectors described in ISO-80369-7:2016, revised in 2021 entitled “Small-bore connectors for liquids and gases in healthcare applications—Part 7: Connectors for intravascular or hypodermic applications”. As shown in FIG. 1, the syringe 12 comprises a syringe body 14 defining a chamber 16 for holding a pharmaceutical composition and having a syringe tip 18 having lumen 20 with an opening 22 through which the pharmaceutical composition can be expelled from the chamber 16. The syringe tip 18 may be, for example, a male luer taper, and may be surrounded by a threaded luer lock 24. The syringe tip 18 is removably attached to the needle hub 26 of a needle assembly 28. A needle 30 having a lumen 32 in fluid communication with an interior volume 34 of the needle hub 26 extends from the needle hub 26. A plunger rod (not shown) attached to a stopper (not shown) disposed within the chamber 16 of the syringe body 14 is movable with respect to the syringe body 14 such that a distal force applied to the plunger rod while holding the syringe body 14 stationary causes the stopper attached thereto to be displaced in a distal direction within the chamber 16 of the syringe body 14, thereby expelling the pharmaceutical composition through the opening 22 of the syringe tip 18, the needle hub 26, and the lumen 32 of the needle 30.

At the end of injection, when the stopper reaches the proximal end of the chamber 16 of the syringe body 14, the pharmaceutical composition remaining in the lumen 20 of the syringe tip 18 and the interior volume 34 of the needle hub 26 cannot be expelled from the syringe 12 no matter how high a force is exerted on the plunger rod and the stopper. This wasted pharmaceutical composition is commonly referred to as the “dead volume” of the syringe 12. The adaptor 10, 10a, 10b acts to reduce the dead volume in a standard syringe 12.

As shown in FIGS. 2-7, the adaptor 10, 10a, 10b comprises a shaft 36 and a seal 38, 38a, 38b. The shaft 36 has a proximal end 40, a distal end 42, and a sidewall 44 extending between the proximal end 40 and the distal end 42. The sidewall 44 defines a lumen 46 extending through the shaft 36, such that both the proximal end 40 and the distal end 42 of the shaft 36 include an opening. The shaft 36 may be substantially cylindrical.

The seal 38, 38a, 38b extends from the distal end 42 of the shaft 36.

In one embodiment shown in FIGS. 2 and 3, the seal 38 surrounds the distal end 42 of the shaft 36 and has a proximal end 48, a distal end 50, and a sidewall 52. At the proximal end 48 of the seal 38, the sidewall 52 of the seal 38 has an outer diameter that is greater than an outer diameter of the shaft 36, such that the proximal end 48 of the seal 38 defines a ledge 54 extending radially from the shaft 36. The outer surface 56 of the sidewall 52 of the seal 38 may taper in the distal direction with the largest outer diameter of the seal 38 at the proximal end 48 of the seal 38, and the smallest outer diameter of the seal 38 at the distal end 50 of the seal 38. In this embodiment, the seal 38 is plug shaped. One or more circumferential ridges 58 may be provided on the sealing surface 60 of the seal 38.

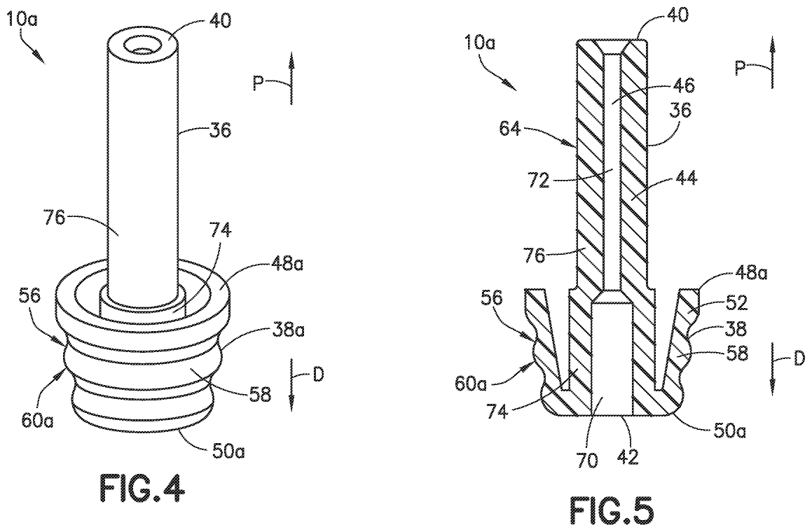

In another embodiment shown in FIGS. 4 and 5, the seal 38a is a circumferential flange extending from the distal end 42 of the shaft 36. The seal 38a may have a concave curvature or the outer diameter of the seal 38a may taper in a distal direction, such that the largest diameter of the seal 38a is at the proximal end 48a of the seal 38a, the smallest diameter of the seal 38a is at the distal end 50a of the seal 38a, and the seal 38a extends in a proximal direction along the shaft 36. In this embodiment, the adaptor 10a is umbrella shaped. One or more circumferential ridges 58 may be provided on the sealing surface 60a of the seal 38a.

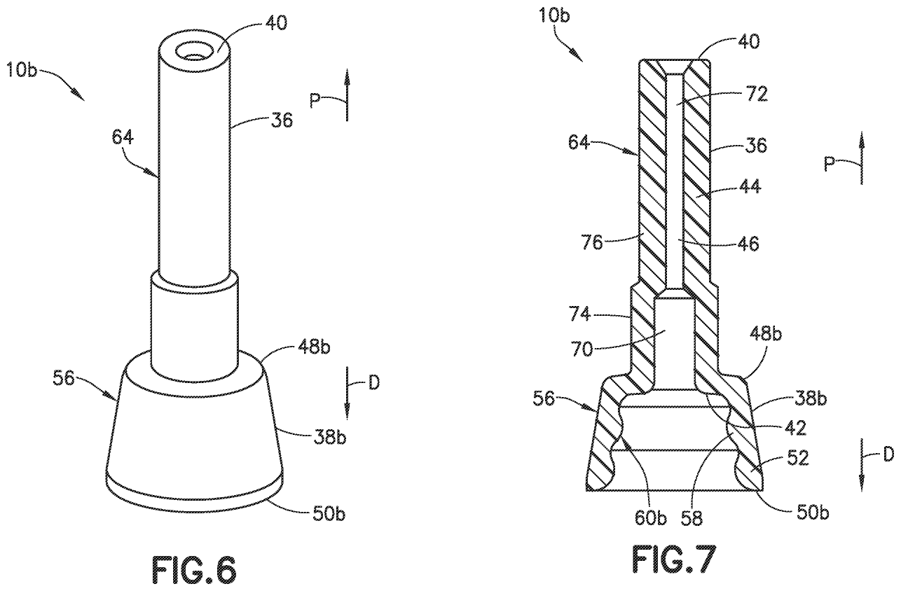

In a further embodiment shown in FIGS. 6 and 7, the seal 38b is a circumferential flange extending from the distal end 42 of the shaft 36. The seal 38b may have a convex curvature or the outer diameter of the seal 38b may taper in a proximal direction, such that the largest diameter of the seal 38b is at the distal end 50b of the seal 38b, the smallest diameter of the seal 38b is at the proximal end 48b of the seal 38b, and the seal 38b extends away from the distal end 42 of the shaft 36. In this embodiment, the adaptor 10b has the shape of an inverted umbrella. One or more circumferential ridges 58 may be provided on the sealing surface 60b of the seal 38b.

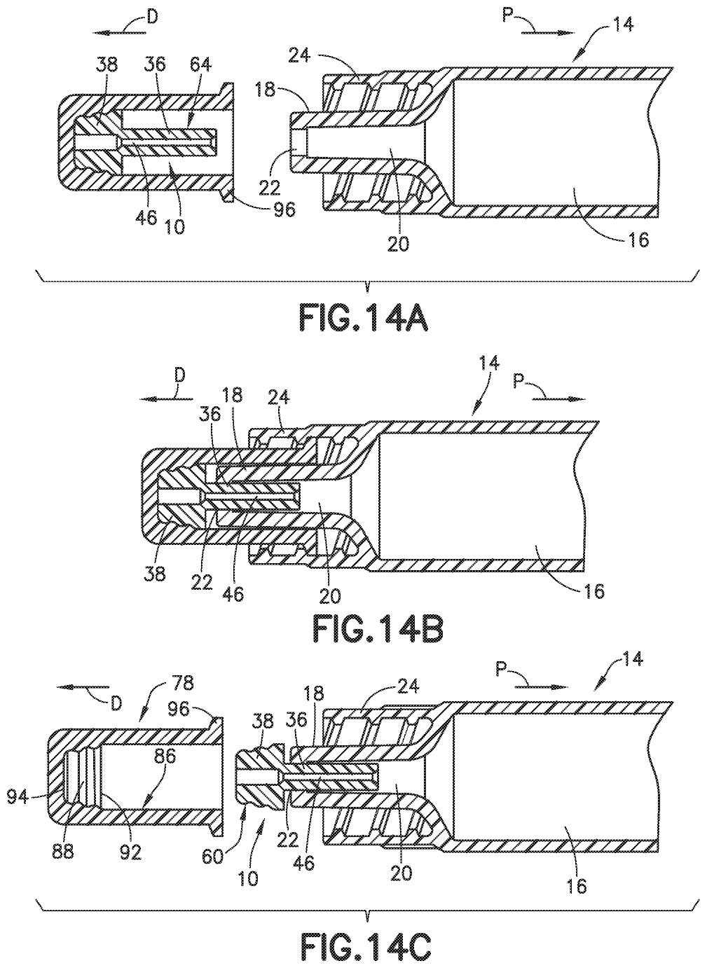

Referring to FIGS. 14A-14C, 15, and 17, at least a portion of the shaft 36 of the adaptor 10, 10a, 10b is sized to be inserted in the lumen 20 of the syringe tip 18. When inserted into the lumen 20 of the syringe tip 18, the shaft 36 of the adaptor 10, 10a, 10b acts to reduce the interior volume 62 of the syringe tip 18 that is filled with medicament, while simultaneously creating a seal with the opening 22 of the syringe tip 18, thereby causing the medicament contained in the syringe 12 to be expelled through the lumen 46 of the shaft 36 of the adaptor 10, 10a, 10b and keeping the medicament from being expelled between the opening 22 of the syringe tip 18 and the outer surface 64 of the shaft 36 of the adaptor 10, 10a, 10b. The length L s of the shaft 36 that is inserted into the lumen 20 of the syringe tip 18 may be substantially equal to the length L t of the lumen 20 of the syringe tip 18.

Referring to FIGS. 16 and 17, the seal 38, 38a, 38b of the adaptor 10, 10a, 10b is sized to be inserted into the needle hub 26 of a needle assembly 28, such that at least a portion of the sealing surface 60, 60a, 60b of the seal 38, 38a, 38b of the adaptor 10, 10a, 10b is in sealing engagement with at least a portion of the inner surface 66 of the sidewall 68 of the needle hub 26 of the needle assembly 28, and the lumen 46 of the shaft 36 of the adaptor 10, 10a, 10b is in fluid communication with the lumen 32 of the needle 30. When inserted into the needle hub 26 of the needle assembly 28, the seal 38, 38a, 38b of the adaptor 10, 10a, 10b creates a seal with the inner surface 66 of the sidewall 68 of the needle hub 26, thereby causing the medicament contained in the syringe 12 to be expelled through the lumen 46 of the shaft 36 of the adaptor 10, 10a, 10b and keeping the medicament from passing around the adaptor 10, 10a, 10b and into the interior volume 62 of the needle hub 26. As a result, the adaptor 10, 10a, 10b reduces the interior volume 62 of the needle hub 26 that is filled with medicament.

The shaft 36 of the adaptor 10, 10a, 10b may be made of any suitable material that creates a fluid-tight seal with the opening 22 of the syringe tip 18. The material may be a material that compresses when the shaft 36 of the adaptor 10, 10a, 10b is inserted into the opening 22 of the syringe tip 18, for example, TPE, rubber, or a mixture of TPE and PE. The shaft 36 of the adaptor 10, 10a, 10b may be held within the opening 22 of the syringe tip 18 and create the seal with the opening 22 of the syringe tip 18 by frictional and/or compressive forces, and may have an outer diameter that is equal to or greater than the inner diameter of the opening 22 of the syringe tip 18.

The sealing surface 60, 60a, 60b of the seal 38, 38a, 38b of the adaptor 10, 10a, 10b, may have any shape that provides a fluid-tight seal with the inner surface 66 of the sidewall 68 of the needle hub 26 of the needle assembly 28. For example, the sealing surface 60, 60a, 60b of the seal 38, 38a, 38b of the adaptor may have the shape of a cone, a truncated cone, a hemisphere, or any other shape that needle hub 26 may have. The seal 38, 38a, 38b of the adaptor 10, 10a, 10b may be made of any suitable material that creates a fluid-tight seal with inner surface 66 of the sidewall 68 of the needle hub 26 of the needle assembly 28. The material may be a material that compresses or flexes when the seal 38, 38a, 38b of the adaptor 10, 10a, 10b is inserted into the needle hub 26 of the needle assembly 28, for example, TPE, rubber, of a mixture of TPE and PE. The seal 38, 38a, 38b may be held within the needle hub 26 of the needle assembly 28 and create the seal with the inner surface 66 of the sidewall 68 of the needle hub 26 of the needle assembly 28 by frictional and/or compressive forces.

The lumen 46 of the shaft 36 of the adaptor 10, 10a, 10b may have a constant diameter or the diameter of the lumen 46 of the shaft 36 of the adaptor 10, 10a, 10b may change along the length of the adaptor 10, 10a, 10b. For example, a distal portion 70 of the lumen 46 of the shaft 36 of the adaptor 10, 10a, 10b that is surrounded by the seal 38, 38a, 38b of the adaptor 10, 10a, 10b and positioned in the needle hub 26 of the needle assembly 28 may have a diameter that is greater than a diameter of a proximal portion 72 of the shaft 36 of the adaptor 10, 10a, 10b that is inserted in the lumen 20 of the syringe tip 18.

The shaft 36 of the adaptor 10, 10a, 10b may have a constant outer diameter or the outer diameter of the shaft 36 of the adaptor 10, 10a, 10b may change along the length of the adaptor 10, 10a, 10b. For example, a distal portion 74 of the shaft 36 of the adaptor 10, 10a, 10b that is surrounded by the seal 38, 38a, 38b of the adaptor 10, 10a, 10b and positioned in the needle hub 26 of the needle assembly 28 may have an outer diameter that is greater than the outer diameter of a proximal portion 76 of the shaft 36 of the adaptor 10, 10a, 10b that is inserted in the lumen 20 of the syringe tip 18.

As shown in FIGS. 8-14C, a delivery housing 78, 78a, 78b, 78c may be provided with the adaptor 10, 10a, 10b to assist in insertion of the adaptor 10, 10a, 10b into either the syringe tip 18 or the needle hub 26 and to prevent the adaptor 10, 10a, 10b from being touched during insertion into the syringe tip 20 or the needle hub 26, thereby maintaining the sterility of the fluid path of the syringe 12. The delivery housing 78, 78a, 78b, 78c comprises an open proximal end 80, a closed distal end 82, and a sidewall 84 extending between the open proximal end 80 and the closed distal end 82. The delivery housing 78, 78a, 78b, 78c may be substantially cylindrical.

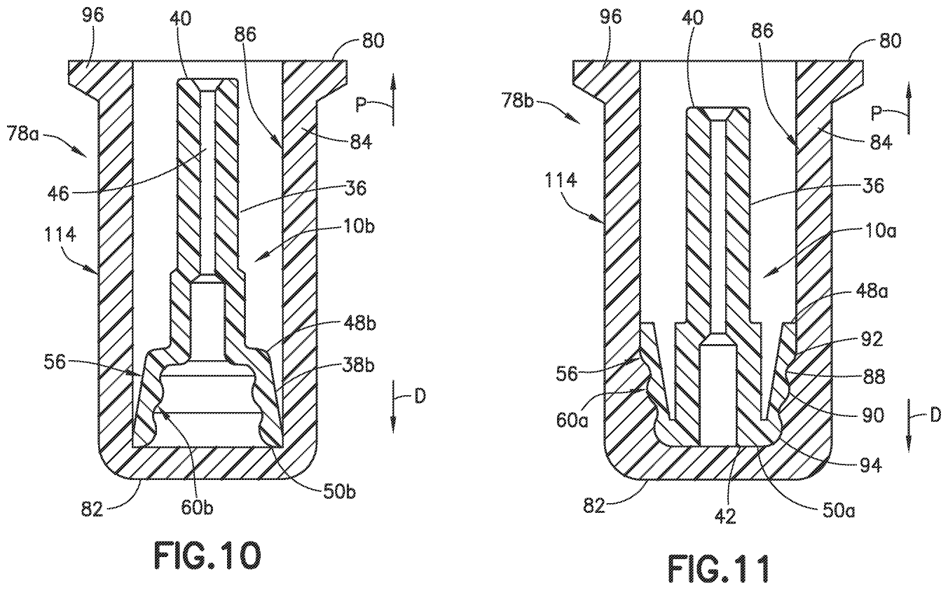

For a delivery housing 78 adapted for inserting the shaft 36 of the adaptor 10, 10a, 10b into the syringe tip 18 (FIGS. 9-11), the inner surface 86 of the sidewall 84 at the closed distal end 82 of the delivery housing 78 may have a diameter equal to or less than the maximum diameter of the seal 38, 38a, 38b, such that the adaptor 10, 10a, 10b is held within the delivery housing 78 by frictional and/or compressive forces between the inner surface 86 of the sidewall 84 of the delivery housing 78 and the seal 38, 38a, 38b (FIGS. 9 and 10).

Alternatively, the inner surface 86 of sidewall 84 of the delivery housing 78a may have a recess 88 having a shape corresponding the shape of the outer surface 56 of the seal 38, 38a, 38b (FIG. 11). The recess 88 may optionally include circumferential grooves 90 for receiving the optional circumferential ridges 58 on the sealing surface 60, 60a, 60b of the seal 38, 38a, 38b. The adaptor 10, 10a, 10b is held within the delivery housing 78a by frictional and/or compressive forces between the recess 88 and the seal 38, 38a, 38b.

In the case where the seal 38, 38a tapers distally or is concave, such that the largest diameter of the seal 38, 38a is at the proximal end 48, 48a of the seal 38, 38a, the smallest diameter of the seal 38, 38a is at the distal end 50, 50a of the seal 38, 38a, and the seal 38, 38a extends in a proximal direction along the shaft 36, the recess 88 in the inner surface 86 of the sidewall 84 of the delivery housing 78a may have a similar shape where the surface of the recess 88 tapers distally, such that the largest diameter of the recess 88 is at the proximal end 92 of the recess 88 and the smallest diameter of the recess 88 is at the distal end 94 of the recess 88 adjacent the closed distal end 82 of the delivery housing 78a.

Optionally, a flange 96 adapted to engage the threads of a luer lock 24 may extend radially outward from the open distal end 82 of the delivery housing 78, 78a.

In use, as shown in FIGS. 14A-14C, the delivery housing 78, 78a is pressed or threaded onto the syringe tip 18 until the shaft 36 of the adaptor 10, 10a, 10b has been inserted into the lumen 20 of the syringe tip 18 (FIG. 14B). The frictional and/or compressive forces between the adaptor 10, 10a, 10b and the delivery housing 78, 78a are less than the frictional and/or compressive forces between the shaft 36 of the adaptor 10, 10a, 10b and the opening 22 in the syringe tip 18, such that the adaptor 10, 10a, 10b remains inserted in the opening 22 of the syringe tip 18 as the delivery housing 78, 78a is removed from the syringe tip 18 (FIG. 14C).

When the syringe tip 18 includes a threaded luer lock 24 and the delivery housing includes a flange 96 extending radially outward from the proximal end 80 of the delivery housing 78, 78a, the delivery housing 78, 78a is threaded into the luer lock 24 to insert the shaft 36 of the adaptor 10, 10a, 10b into the lumen 20 of the syringe tip 18 and unthreaded from the luer lock 24 after insertion.

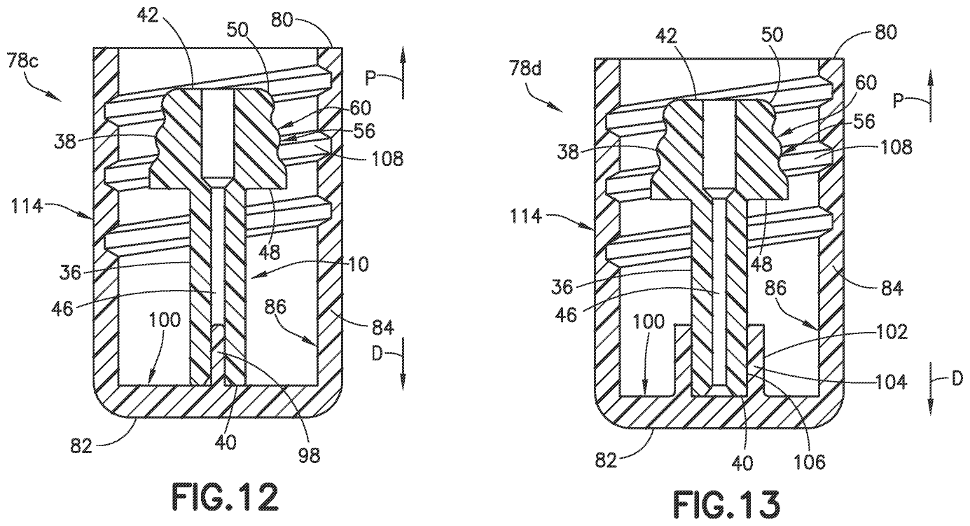

For a delivery housing 78b adapted for inserting the seal 38, 38a, 38b of the adaptor 10, 10a, 10b into the needle hub 26 (FIGS. 12 and 13), a post 98 may extend proximally outward from the inner surface 100 of the closed proximal end 80 of the delivery housing 78b, and the post 98 is inserted into the lumen 46 of the shaft 36 of the adaptor 10, 10a, 10b (FIG. 12). The post 98 may have an outer diameter equal to or greater than the diameter of the lumen 46 of the shaft 36 of the adaptor 10, 10a, 10b, such that the adaptor 10, 10a, 10b is held within the delivery housing 78b by frictional and/or compressive forces between the outer surface of the post 98 and the inner surface of the lumen 46 of the shaft 36.

Alternatively, a protrusion 102 comprising a sidewall 104 defining a passageway 106 may extend proximally outward from the inner surface 100 of the closed proximal end 80 of the delivery housing 78c, and the shaft 36 of the adaptor 10, 10a, 10b is inserted in the passageway 106 of the protrusion 102 (FIG. 13). The passageway 106 of the protrusion 102 may have a diameter equal to or less than the outer diameter of the shaft 36 of the adaptor 10, 10a, 10b, such that the adaptor 10, 10a, 10b is held within the delivery housing 78c by frictional and/or compressive forces between the inner surface of the sidewall 104 of the protrusion 102 and the outer surface of the sidewall 44 of the shaft 36.

Optionally, the delivery housing 78b, 78c may include threads 108 on an inner surface of the sidewall 84 of the delivery housing 78b, 78c.

In use, the delivery housing 78b, 78c is pressed or threaded onto the needle hub 26 until the seal 38, 38a, 38b of the adaptor 10, 10a, 10b has been inserted into the interior volume 62 of the needle hub 26 and is in sealing engagement with the inner surface 66 of the sidewall 68 of the needle hub 26. The frictional and/or compressive forces between the adaptor 10, 10a, 10b and the delivery housing 78b, 78c are less than the frictional and/or compressive forces between the seal 38, 38a, 38b of the adaptor 10, 10a, 10b and the inner surface 66 of the sidewall 68 of the needle hub 26, such that the adaptor 10, 10a, 10b remains inserted in the needle hub 26 as the delivery housing 78b, 78c is removed from the needle hub 26.

When the delivery housing 78b, 78c includes threads 108 on the inner surface 86 of the sidewall 84 and the needle hub 26 includes a flange 110 extending radially outward from the proximal end 106 of the needle hub 26, the needle hub 26 is threaded into the delivery housing 78b, 78c to insert the adaptor 10, 10a, 10b into the needle hub 26 and unthreaded from the delivery housing 78b, 78c after insertion.

In the case where the seal 38b of the adaptor 10b is a flange that in the initial position has an outer surface 56 that tapers proximally or is convex, i.e., the adaptor 10b has the shape of an inverted umbrella, as the needle hub 26 is pressed or threaded into the needle hub 26, the seal collapses from the initial position to a position in which the seal 38b has reversed direction such that the sealing surface 60b is in contact with the inner surface 66 of the sidewall 68 of the needle hub 26 and the seal 38b has a shape that tapers proximally or has a concave shape, i.e., the adaptor 10b is umbrella shaped.

Optionally, any of the delivery housings 78, 78a, 78b, 78c may include ridges 112 extending radially outward from the outer surface 114 of the sidewall 84 of the delivery housing 78, 78a, 78b, 78c to assist the user in manipulating the delivery housing 78, 78a, 78b, 78c. The ridges 112 may extend longitudinally along the sidewall 84 of the delivery housing 78, 78a, 78b, 78c.

In use, as shown in FIG. 17, after the shaft 36 of the adaptor 10, 10a, 10b is inserted into the lumen 20 of the syringe tip 18 and seals with the opening 22 of the syringe tip 18 or the seal 38, 38a, 38b of the adaptor 10, 10a, 10b is inserted in the needle hub 26 and seals with the inner surface 66 of the sidewall 68 of the needle hub 26, the syringe tip 20 is then inserted into and connected to the needle hub 26, such that the seal 38, 38a, 38b of the adaptor 10, 10a, 10b is inserted into the needle hub 26 and seals with the inner surface 66 of the sidewall 68 of the needle hub 28 or the shaft 36 of the adaptor 10, 10a, 10b is inserted into the lumen 20 of the syringe tip 18 and seals with the opening 22 of the syringe tip 20, respectively.

If the syringe tip 20 is provided with a threaded luer lock 24 and the needle hub 26 is provided with a proximal flange 110, attachment of the needle hub 26 to the syringe tip 20 by threading the needle hub 26 onto the syringe tip 18 causes the shaft 36 of the adaptor 10, 10a, 10b to be inserted into the lumen 20 of the syringe tip 18 and seal with the opening 22 of the syringe tip 18 or the seal 38, 38a, 38b of the adaptor 10, 10a, 10b to be inserted in the needle hub 26 and seal with the inner surface 66 of the sidewall 68 of the needle hub 26. Further tightening of the connection assures that good seals are obtained between the shaft 36 of the adaptor 10, 10a, 10b and the opening 22 of the syringe tip 18 and between the seal 38, 38a, 38b of the adaptor 10, 10a, 10b and the inner surface 66 of the sidewall 68 of the needle hub 26 and that the shaft 36 of the adaptor 10, 10a, 10b extends into the syringe tip 18.

The adaptor 10, 10a, 10b may be attached to or provided with any standard syringe 12, for example, ISO luer compliant syringes. The adaptor may be provided as a separate part that is attached to the syringe body 14 and needle hub 26 by a medical practitioner at the point of care (FIGS. 2-7), the adaptor 10, 10a, 10b may be attached to syringe bodies 14 during manufacturing for later attachment to a needle hub 26 (FIG. 15), to needle hubs 26 during manufacturing for later attachment to syringe bodies 14 (FIG. 16), or may be provided within an already assembled syringe body 14 and needle hub 26 (FIG. 17).

As can be seen in FIG. 1, with standardized syringe bodies 14 and needle assemblies 28 that utilize needle assemblies 28 having needle hubs 26 that connect to a syringe tip 18 (luer taper), the interior volume 62 of the lumen 20 of the syringe tip 18 and the interior volume 34 of the needle hub 26 are large, such that a significant amount of medicament is retained in these areas after an injection using the syringe 12. This volume of medicament as well as the medicament that is retained in the lumen 32 of the needle 30 is referred to as the “dead volume”. The inventive adaptor 10, 10a, 10b adapts traditional, standardized syringe bodies and needle assemblies to deliver a low dead volume benefit without purchasing dedicated or specialized low dead volume devices.

At least a portion of the shaft 36 of the adaptor 10, 10a, 10b fills part of the interior volume 62 of the lumen 20 of the syringe tip 18 and the seal 38, 38a, 38b of the adaptor 10, 10a, 10b fills and isolates a portion of the interior volume 34 of the needle hub 26 that would otherwise be filled with the medicament, thereby reducing the dead volume of the standard syringe 12. By reducing the dead volume of the syringe 12, the adaptor 10, 10a, 10b reduces the amount of medicament that is wasted, thereby saving money and allowing more injections to be provided from a given volume of medicament.

Whereas particular aspects of this invention have been described above for purposes of illustration, it will be evident to those skilled in the art that numerous variations of the details of the present invention may be made without departing from the invention.

The invention claimed is:

1. An adaptor for a syringe having a syringe tip defining a lumen with an opening and a needle assembly having a needle hub attached to the syringe tip, the adaptor comprising:
a shaft having a proximal end, a distal end, and a sidewall extending between the proximal end and the distal end and defining a lumen extending through the shaft; and
a seal that extends from the distal end of the shaft,
wherein at least a portion of the shaft is sized and shaped to be inserted in the lumen of the syringe tip and create a seal with the opening of the syringe tip, and at least a portion of the seal is sized and shaped to be inserted into the needle hub of the needle assembly to create a seal with an inner surface of the needle hub, thereby reducing an interior volume of the lumen of the syringe tip and an interior volume of the needle hub that is filled with medicament contained in the syringe.

2. The adaptor of claim 1, wherein the seal surrounds the distal end of the shaft and has a proximal end, a distal end, and a sidewall, and an outer surface of the sidewall of the seal tapers in the distal direction with a largest outer diameter of the seal at the proximal end of the seal, and a smallest outer diameter of the seal at the distal end of the seal.

3. The adaptor of claim 1, wherein the seal is a circumferential flange extending from the distal end of the shaft.

4. The adaptor of claim 3, wherein the seal has a concave curvature or the outer diameter of the seal tapers in a distal direction, such that a largest diameter of the seal is at a proximal end of the seal, a smallest diameter of the seal is at a distal end of the seal, and the seal extends in a proximal direction along the shaft.

5. The adaptor of claim 1, wherein the seal is a circumferential flange extending from the distal end of the shaft, the seal has a convex curvature or the outer diameter of the seal tapers in a proximal direction, such that the largest diameter of the seal is at a distal end of the seal, the smallest diameter of the seal is at a proximal end of the seal, and the seal extends away from the distal end of the shaft.

6. The adaptor of claim 1, wherein the shaft and/or the seal is made of a material that compresses or flexes when the shaft is inserted into the opening of the syringe tip or the seal is inserted in the needle hub, respectively.

7. The adaptor of claim 1, wherein a sealing surface of the seal has the shape of a cone, a truncated cone, a hemisphere, or a cylinder with a front portion having a flat seal surface.

8. The adaptor of claim 1, wherein a diameter of the lumen of the shaft changes along the length of the adaptor.

9. The adaptor of claim 7, wherein a distal portion of the lumen of the shaft that is surrounded by the seal and positioned in the needle hub has a diameter that is greater than a diameter of a proximal portion of the shaft that is inserted in the lumen of the syringe tip.

10. The adaptor of claim 1, wherein a distal portion of the shaft that is surrounded by the seal and positioned in the needle hub has an outer diameter that is greater than an outer diameter of a proximal portion of the shaft that is inserted in the lumen of the syringe tip.

11. An adaptor assembly comprising:
an adaptor according to claim 1; and
a delivery housing to which the adaptor is attached,
wherein the delivery housing comprises an open proximal end, a closed distal end, and a sidewall extending between the proximal end and the distal end.

12. The adaptor assembly of claim 11, wherein an inner surface of the sidewall of the delivery housing at the closed distal end of the delivery housing has a diameter equal to or less than a maximum diameter of the seal, such that the adaptor is held within the delivery housing by frictional and/or compressive forces between the inner surface of the sidewall of the delivery housing and the seal.

13. The adaptor assembly of claim 12, wherein frictional and/or compressive forces between the adaptor and the delivery housing are less than the frictional and/or compressive forces between the seal of the adaptor and the inner surface of the sidewall of the needle hub.

14. The adaptor assembly of claim 11, wherein an inner sidewall of the delivery housing has a recess having a shape corresponding to a shape of the outer surface of the seal.

15. The adaptor assembly of claim 14, wherein frictional and/or compressive forces between the adaptor and the delivery housing are less than the frictional and/or compressive forces between the seal of the adaptor and the inner surface of the sidewall of the needle hub.

16. The adaptor assembly of claim 11, wherein a post extends proximally outward from an inner surface of the closed proximal end of the delivery housing and the post is inserted in the lumen of the shaft of the adaptor.

17. The adaptor assembly of claim 16, wherein frictional and/or compressive forces between the adaptor and the delivery housing are less than the frictional and/or compressive forces between the shaft of the adaptor and the post.

18. The adaptor assembly of claim 11, wherein a protrusion comprising a sidewall defining a passageway extends proximally outward from an inner surface of the closed proximal end of the delivery housing and the shaft of the adaptor is inserted in the passageway of the protrusion.

19. The adaptor assembly of claim 18, wherein frictional and/or compressive forces between the adaptor and the delivery housing are less than the frictional and/or compressive forces between the shaft of the adaptor and the protrusion.

20. The adaptor assembly of claim 11, wherein a flange adapted to engage threads of a luer lock on the syringe extend radially outward from the open distal end of the delivery housing.

21. The adaptor assembly of claim 11, wherein the delivery housing includes threads on an inner surface of the sidewall

22. The adaptor assembly of claim 11, wherein the delivery housing includes ridges extending radially outward from an outer surface of the sidewall.

23. A syringe body comprising:
a chamber having a syringe tip defining a lumen having an opening through which medicament may be expelled from the chamber;
and an adaptor according to claim 1,
wherein at least a portion of the shaft of the adaptor is positioned in the lumen of the syringe tip and forms a seal with the opening of the lumen.

24. The syringe body of claim 23, wherein a length of the shaft that is inserted into the lumen of the syringe tip is substantially equal to a length of the lumen of the syringe tip.

25. A needle assembly comprising:
a needle hub having a sidewall defining an interior volume;
a needle having a lumen in fluid communication with the interior volume of the needle hub and extending from the needle hub; and
an adaptor according to claim 1,
wherein the seal of the adaptor is positioned in the interior volume of the needle hub and forms a seal with an inner surface of the sidewall of the needle hub.

26. A syringe comprising:
a syringe body comprising a chamber having a syringe tip defining a lumen having an opening through which medicament may be expelled from the chamber;
a needle hub having a sidewall defining an interior volume connected to the syringe tip;
a needle having a lumen in fluid communication with the interior volume of the needle hub and extending from the needle hub; and
an adaptor according to claim 1,
wherein at least a portion of the shaft of the adaptor is positioned in the lumen of the syringe tip and forms a seal with the opening of the lumen, the seal of the adaptor is positioned in the interior volume of the needle hub and forms a seal with an inner surface of the sidewall of the needle hub, and the lumen of the shaft of the adaptor is in fluid communication with the chamber and the lumen of the needle.

27. The syringe of claim 26, wherein at least a portion of the shaft of the adaptor fills part of a volume of the lumen of the syringe tip and the seal of the adaptor fills and isolates a portion of the interior volume of the needle hub that would otherwise be in filled with the medicament.

28. The syringe of claim 26, wherein a length of the shaft that is inserted into the lumen of the syringe tip is substantially equal to a length of the lumen of the syringe tip.

29. A method of reducing the dead volume of a syringe having a syringe tip defining a lumen with an opening and a needle assembly having a needle hub attached to the syringe tip, the method comprising:
providing an adaptor according to claim 1;
inserting at least a portion of the shaft of the adaptor in the lumen of the syringe tip; and
inserting the seal of the adaptor in an interior volume of the needle hub,
wherein a seal is provided between the shaft of the adaptor and the opening of the syringe tip, and a seal is provided between the seal of the adaptor and an inner surface of the needle hub, and
the adaptor reduces an interior volume of the lumen of the syringe tip and the interior volume of the needle hub that is filled with medicament contained in the syringe.
